(19) United States
(12) Patent Application Publication
(10) Pub. No.: US 2004/0136904 A1
(43) Pub. Date: Jul. 15, 2004

(54) Dry powder compositions comprising labelled particles for inhalation

(76) Inventor: Gary R. Pitcairn, Nottingham (GB)

Correspondence Address: RYAN KROMHOLZ & MANION, S.C., POST OFFICE BOX 26618, MILWAUKEE, WI, 53226, US

(21) Appl. No.: 10/469,894

(22) PCT Filed: Mar. 13, 2002
(86) PCT No.: PCT/GB02/01148

(30) Foreign Application Priority Data
Mar. 15, 2001 (GB) 0106403.9

(51) Int. Cl. A61K 51/00; A61L 9/04; A61K 9/14

(52) U.S. Cl. 424/1.11; 424/46

(57) ABSTRACT

A dry powder composition is described. In one embodiment, the composition comprises smaller label particles attached to larger particles. In another embodiment, the composition comprises label particles comprising a core of a gamma emitting radionuclide and a shell of a non-radioactive material that are attached to a second particulate material. A method for preparing the compositions as well as their use in the evaluation of drug distribution following inhalation of the compositions into the respiratory tract is also described.

The present invention relates to powder formulations for the delivery of drugs by inhalation. More particularly, the present invention relates to labelling such formulations

Powder formulations can be used for the delivery of drugs to the lung as well as to the nasal cavity. Such formulations can be used to provide a local effect or to deliver the drug into the systemic circulation. In order to achieve an appropriate effect, the particle size of the drug powder formulation and the powder properties need to be optimised so that a maximum quantity of the drug reaches the appropriate site within the respiratory tract. This area has been well reviewed. Various books, for example, Inhalation Delivery of Therapeutic Peptides and Proteins (A L Adjci and P K Gupta, eds), Marcel Dekker, New York, 1997, Aerosols and the Lung: Clinical and Experimental Aspects (S W Clarke and D Pavia, eds), Butterworths, London, 1984, Respiratory Drug Delivery, (P R Byron, ed.), CRC Press, 1990 and Aerosols In Medicine—Principles, Diagnosis and Therapy, 2^{nd} revised edition (F Morén, M B Dolovich, M T Newhouse, S P Newman, eds.), Elsevier, Amsterdam, 1993. For example, in order to get good deposition in the lung, the particle size of a powder formulation needs to be less than about 5 μm as determined by the aerodynamic diameter. Good deposition in the nasal cavity can be achieved with particles that have a size greater than 10 μm aerodynamic diameter

The properties of a candidate dry powder formulation for lung delivery can be evaluated in vitro using a particle size classification apparatus such as an impactor or impinger. Full details of such devices can be found in the book edited by Purewal and Grant (Washington, C., Particle Size Analysis in Inhalation Therapy in Metered Dose Inhaler Technology, Interpharm Press, Inc. Illinois, 1998). The quantity of powder less than 5 μm in size has been referred to as the respirable fraction and is often quoted as a percentage.

A wide variety of dry powder delivery devices are currently available and examples include, Spinhaler (Fisons, Rochester, N.Y.), Rotahaler (Glaxo Wellcome, Research Triangle Park, N.C.), Inhalator (Boehringer Ingelheim, Ridgfield, Conn.), Diskhaler (GlaxoWellcome), Turbohaler (Astra Pharmaceuticals, Lund, Sweden). Further details can be found in the chapter by Dalby et al., “Medical Devices for the Delivery of Therapeutic Aerosols to the Lungs” in the book, Inhalation Aerosols Physical and Biological Basis for Therapy, (A J Hickey, ed.), Marcel Dekker, Inc., New York, 1996, p.441. The process of the patient taking a breath activates some of these systems, whereas some, particularly the system from Inhale Corporation, California, USA, is in the form of a “standing cloud” that the patient inhales. Some devices have an active process in which the powder can be well dispersed, thereby allowing maximum lung deposition. For example, the device available from Dura (San Diego, Calif.), employs a small motor to disperse the powder.

Recently, the use of dry powder formulations for the delivery of drugs via the lungs into the systemic circulation has been described (Patton et al., Pulmonary Delivery of Peptides and Proteins for Systemic Action, Adv. Drug Del. Rev 8 179-196 (1992); insulin has been given particular attention.

It is important when developing a powder device for pulmonary or nasal administration, that the performance of the system is evaluated in human subjects in the form of a lung or nasal deposition study. For a lung deposition study, typically, a labelled formulation is inhaled and the deposition of the powder in the different regions of the respiratory tract (as well as material that impacts the throat and is swallowed, and material remaining within the delivery device or within a spacer system intended to improve delivery) are then quantified. The standard process in such studies involves the labelling of the formulation with a gamma emitting radionuclide such as technetium-99m. This radionuclide has a suitable energy for visualisation of the different regions of interest on a standard gamma camera and the half-life is such that the volunteer or patient taking part in a study is not exposed to hazardous levels of radiation.

The prior art literature contains many examples where dry powder formulations have been so labelled and their lung deposition has been evaluated (S. P. Newman, Therapeutic Aerosol Deposition in Man in Aerosols in Medicine, Principles, Diagnosis and Therapy (F Morén, M B Dolovich, M T Newhouse and S P Newman (eds.), Elsevier Science Publishers D. V., Holland, 1993, p.375)). Typically for a dry powder system, 10-20% of the dose is found in the lung and of this material, about 50% is found in the deep lung (the alveolar region) while the remainder is within the central lung. For the delivery of peptides and proteins, it is considered essential that a large quantity of the drug is delivered to the deep lung.

While it is possible to administer the drug in a powder form without any additional pharmaceutical additives or excipients, it is often the case that the drug is admixed with an inert carrier, such as lactose. A typical procedure is to take fine drug particles (particles less than 5 μm) and coarse carrier particles (greater than 20 μm) that are then mixed. The small drug particles adhere to the larger carrier particles. When this system is administered to the human lung using a suitable administration device, disassociation of the fine particles from the larger carrier particles occurs, such that the fine particles find their way into the lung whereas the larger particles impact in the mouth or on the back of the throat. This formulation process using coarse carrier particles and small adherent drug particles has been well described in the book edited by Zeng and others (Particulate Interactions in Dry Powder Formulations for Inhalation, Taylor and Francis, London, 2001).

The labelling of a dry powder formulation for inhalation with a gamma emitting radionuclide, is not a simple procedure. It is important that the labelled formulation has comparable characteristics to the original unlabelled drug formulation in order that the results obtained in a gamma scintigraphic study have validity. Therefore, workers in the field need to spend considerable time and effort in labelling such dry powder formulations. A standard procedure has been described by Pitcairu and Newman (Radiolabelling of dry powder formulations in Respiratory Drug Delivery VI (R Dalby, I Byron and S Farr (eds.), Interpharm Press Inc. Buffalo Grove, Ill., 1998, p 397). Technetium-99m in the form of pertechnetate is obtained from a standard generator system. The pertechnetate is then extracted into a suitable organic solvent such as pentanol or butanol. This solvent is then removed to give a dry residue in a suitable container, for example in a conical flask. This dry residue is then taken up in a non-solvent such as a hydrofluorocarbon and admixed with the drug powder. Following removal of the non-solvent, the radiolabelled drug is mixed with excipients (if required) and the resulting powder blend filled into a dry powder inhaler for testing. It has been found that it is critical for good labelling that each stage in this process is well defined and controlled, otherwise the particle size of the labelled drug particles does not conform well with the particle size of the unlabelled drug particles as measured for example using an Anderson cascade impactor.

Factors such as the water content in the chosen organic solvent, mixing procedures, use of ion pairing agents, etc. have all been shown to have a critical influence. The sensitivity of the radiolabelling process to these factors means that numerous replicates have to be performed as the control procedures are developed and implemented. Moreover, because of the relatively short half-life of technetium-99m, it is essential to begin the labelling work with a high level of radioactivity. Consequently, those workers charged with the multi-step manufacture of such labelled formulations are frequently exposed to high levels of radiation.

Thus, it would be extremely beneficial if a simpler procedure for the labelling of dry powder formulations for inhalation were available. It is, therefore, an object of the present invention to provide an alternative process for the labelling of dry powder formulations for inhalation.

The present applicant has discovered a new labelled dry powder formulation and a new method for labelling dry powder formulations.

According to the present invention there is provided a dry powder composition which comprises small label particles that are attached to a second particulate material, typically of larger particles. The composition is suitable for administration to the respiratory tract of a mammal such as man by a process of inhalation.

A second aspect of the present invention provides a dry powder composition comprising label particles having a core of a gamma emitting radionuclide and a shell of a non-radioactive material that are attached to a second particulate material.

A third aspect of the present invention provides a method for radiolabelling a material composed of larger particles in which smaller labelled particles are adhered to the larger particles to form a labelled composition.

A further aspect of the present invention provides a method for radiolabelling a particulate material in which label particles comprising a core of a gamma emitting radionuclide and a shell of a non-radioactive material are adhered to the particles of the particulate material to form a labelled composition.

A still further aspect of the present invention provides a method for evaluating the distribution of a material inhaled into the respiratory tract. The method comprises using a dry powder composition comprising smaller label particles that are attached to larger particles of the material to be evaluated. The labelled particles are then delivered to the respiratory tract, e.g. using a dry powder delivery device.

In yet another aspect of the present invention there is provided a method for evaluating the distribution of a particulate material inhaled into the respiratory tract. The method comprises associating with said particulate material label particles comprising a core of a gamma emitting radionuclide and a shell of a non-radioactive material. The labelled particulate material is then delivered to the respiratory tract, e.g. using a dry powder delivery device.

The present invention also provides for the use of label particles comprising a core of a gamma emitting radionuclide and a shell of a non-radioactive material to evaluate the distribution of a second particulate material in the respiratory tract.

In a preferred embodiment, the composition is specifically adapted for delivery to either the lung or the nasal cavity.

The label particles, e.g. radioactive nanoparticles, that are used in the powder compositions of the present invention should be inert under the conditions which are used to deliver those compositions to the respiratory tract. By inert, we mean that the label particles do not significantly affect the deposition profile of the powder composition in the respiratory tract or the delivery of a drug to the systemic circulation through absorption.

Label particles that are suitable for use in the present invention include particles that are radiolabelled with a gamma emitting radionuclide such as technetium-99m, iodine-123 and indium-111. A preferred radiolabel is technetium-99m. The label particles may be entirely composed of the radiolabel or they be partially composed of the radiolabel and some other material. A preferred label particle comprises a central region or core of a radiolabel and a shell that at least partially and preferably completely surrounds or encases the radiolabel core. The shell is made of a material that is not radioactive.

In a preferred embodiment, the dry powder formulation of the present invention is labelled using radioactive nanoparticles.

By nanoparticles, we are particularly referring to solid particles that have a mean particle size between 1 nm and 200 nm and preferably of less than 100 nm, e.g. between 1 nm and 100 nm. By the size of a particle, we are referring to the diameter of the particle where the particle is spherical and to the size across the largest dimension of the particle where the particle is irregularly shaped.

Preferred nanoparticles for use in the present invention are those comprising technetium-99m.

In a preferred embodiment, the nanoparticle comprises a central region or core of the radiolabel and a shell that at least partially and preferably completely surrounds or encases the radiolabel core. The shell is made of a material that is not radioactive. A preferred material for the shell is carbon.

It will be appreciated that nanoparticles comprising an outer carbon shell and an inner core of another gamma emitting radionuclide such as iodine 123 or indium-111 are also useful in the present invention as are nanoparticles having a shell made of a non-radioactive material other than carbon.

The preferred nanoparticles for labelling are the Technegas and Pertechnegas particles produced by a Technegas generator.

Technegas particles comprise hexagonal flat crystals of technetium-99m encased in multiple layers of carbon such that the technetium 99m metal is protected from the environment, thus preventing it from oxidising and forming pertechnetate.

Pertechnegas particles are similar to Technegas particles except that the technetium 99m is not completely encased by carbon. Consequently, Pertechnegas particles form pertechnetate once exposed to trace levels of oxygen in the aerosol.

The Technegas generator is commercially available (Qados Ltd, Unit 8, Lakeside Business Park, Swan Lane, Sandhurst, Berkshire) and consists of a high temperature furnace containing a heating element in the form of a carbon crucible which provides the graphite vapour that coats the technetium-99m metal.

The standard procedure for producing Technegas is as follows:

Sodium pertechnetate in saline solution, collected from a standard elution generator, such as Elumatic 3 available from Schering Healthcare, is loaded into a carbon crucible and placed inside the Technegas generator chamber. The chamber is sealed and the water in the sodium pertechnetate solution removed by heating the crucible to 70° C. in a pure argon atmosphere. The chamber is then purged with pure argon to remove air and water vapour and the crucible is resistively heated to approximately 2550° C. to produce primary Technegas particles suspended in argon. The primary Technegas particles are 5 to 30 nm in size (i.e. across the hexagonal face) and approximately 3 nm thick. These primary particles rapidly form agglomerates with an average size of 100 nm.

The same procedure is used to produce Pertechnegas particles, except that the argon supply used contains greater than 0.2% by volume oxygen.

The second particulate material in the dry powder compositions of the invention is a carrier for the label particles and is normally comprised of larger particles. Typically, the carrier particles have a mean particle size of between 500 nm and 100 μm. Preferably, the carrier particles have a mean size of between 1 μm and 10 μm and more preferably are drug particles. The size of the particles will depend on their intended route of delivery (nose, lung).

Drugs that can be labelled in accordance with the present invention include all drugs formulated for administration to the respiratory tract of a mammal by a process of inhalation. These include beta-2-agonists such as salbutamol, anticholingeries such as ipratropium bromide, corticosteroids such as budesonide, non-steroidal anti-inflammatory agents such as sodium cromoglycate and nedocromil sodium, polypeptides, insulin, growth hormones, parathyroid hormone, calcitonin, octreotide, leuprolide, leutenising releasing hormone, alpha, beta and gamma interferons, aerosolised antibiotics such as gentamicin, anti-infectives such as pentamidine, anti-virals such as rimantadine, mucolytic agents such as rhDNase, alpha-1-antirypsin, diuretics such as fiusemide, phospodiesterase inhibitors and leukotriene antagonists.

In order to produce the powder composition of the present invention, the label particles (e.g. nanoparticles) are adhered to the carrier particles (e.g. drug particles) that are intended for delivery to the respiratory tract, e.g. nose or lung.

The label particles emitted from a Technegas generator (both Technegas and Pertechnegas particles) may be readily adhered to larger carrier particles such as drug particles by directing the aerosol from the generator through a dry bed of powder to be labelled. The amount of label particles that adhere to the carrier particles can be controlled by varying the total volume of the Technegas/Pertechnegas particle stream.

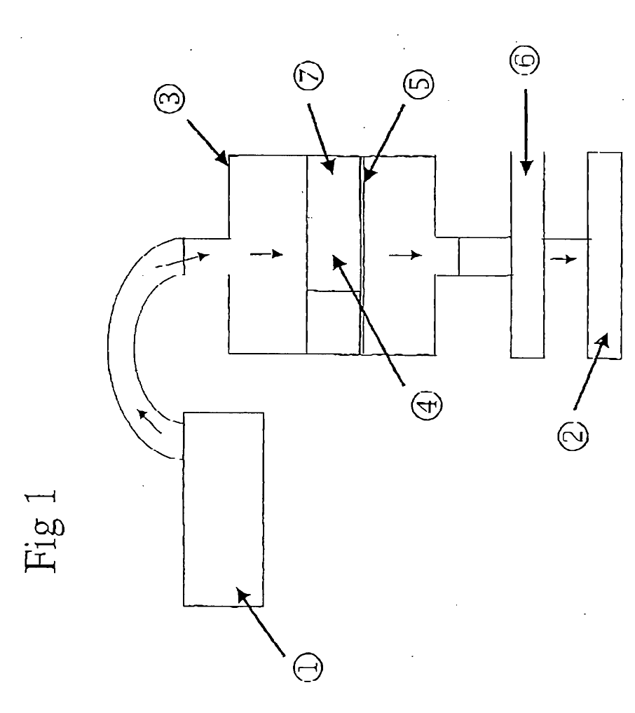

FIG. 1 illustrates a filter assembly apparatus that is connected to a Technegas generator (1) for radiolabelling carrier particles in the dry powder state.

The Technegas particle stream is drawn out of the generator (1) by means of a vacuum pump (2) and through a chamber (3) containing the powdered carrier material (4) supported on a piece of filter paper (5). The apparatus is arranged such that the Technegas particle stream has to pass through the powder bed (4) and out of the chamber through a filter (6).

This process is preferably repeated, in which case the powder is removed from the die (7), mixed using a metal spatula and replaced in the apparatus. Another Technegas particle stream is then drawn through the powder.

It will be appreciated that the same filter apparatus could be used to label dry, powdered carrier particles with other radiolabels.

In another labelling procedure, the label particles are first captured in a solvent which is a non-solvent for the carrier particles to be labelled by slowly bubbling the Technegas/Pertechnegas particle stream through the solvent. Adding the carrier particles to the non-solvent containing the label particles and then removal of the non-solvent will produce the labelled particles of the present invention. The non-solvent which is used will, of course, depend on the composition of the carrier particles. Suitable non-solvents may be selected from the hydrofluorocarbons

It will appreciated by those skilled in the art that the above procedures for labelling particles that have been described with reference to Technegas/Pertechnegas particles produced by the Technegas generator can be readily modified to use radiolabels other than technetium-99m.

The person skilled in the art will also appreciate that in order to achieve good adhesion between the smaller label particles and the larger carrier particles, the physicochemical properties of the two particulate systems may need to be taken into account.

In some cases, good adhesion may be achieved through simple Van der Waals interactions (Zeng et al. 2001), but in other cases it may be necessary to increase the attraction between the two different particles. To this end, it is possible to exploit the methods known in the field of electrodeposition, for example the charge on the label particles can be different to that on the carrier particles so that strong adherence between the particles is achieved through electrostatic interaction.

It is known from the prior art that the charge on drug particles can be controlled through pharmaceutical processing, for example, Carter et al. (An Experimental Investigation of Triboelectrification in Cohesive and Non-cohesive Pharmaceutical Powders, Drug Dev. Ind. Pharm. 18 1505 (1991)), have shown that the charge carried on salbutamol and beclomethasone dipropionate can be altered by the nature of the vessel used to contain the powder during processing involving fluidisation. With salbutamol, a brass vessel led to the particles having a positive charge whereas a steel vessel led to the particles having a negative charge. The opposite effect was found for beclomethasone dipropionate.

The properties of the nanoparticles produced by the Technegas generator can be further modified if required using a particle static charging process.

Other methods for improving the adhesion between particles could also be considered. Particle particle adhesion in pharmaceutical powder handling has been well described by Podezeck in a monograph of that title, 1998, Imperial College Press, London. The forces causing particle-particle adhesion include Lifshitz-Van der Waals forces, electrical double-layer forces and electrostatic (Coulomb) forces. Factors influencing adhesion include surface roughness and shape. Lifshitz-Van der Waals forces are about ten times larger than electrical double-layer forces and Coulomb forces. After an adhesion contact has been formed, Lifshitz-Van der Waals forces are responsible for adhesion strength and Coulomb forces become of secondary importance. The importance of electrostatic interaction in aerosol systems has been well considered by Byron et al, Pharm. Res. 14, 698-705 (1997) and Peat et al, Inst. Phys. Conf. Series 143, 271-274 (1995).

Podezech (p. 116) has also considered in detail the re-suspension of dry powder inhalations and the forces acting on a particle adhered to a carrier particle when placed in an air stream. The adhesion force in an interactive powder mixture can be controlled in several ways, to include particle size, shape and surface roughness. For example a decrease in median particle size increases the adhesion force between a small particle and a larger carrier particle. Larger forces of adhesion between particles can be obtained for irregularly shaped or elongated particles. Staniforth (Proc. Drug Delivery to the Lung VII, The Aerosol Soc., London, 1996, p 86-89), has described a corrasion mechanism (the filling of grooves and clefs of larger particles by smaller particles).

The choice of carrier also influences the strength of the adhesion force, possibly due to the effect on moisture adsorption. The person skilled in the art will, if necessary, be able to undertake suitable experiments to augment the adhesion between the label particles and the carrier particles taking into account the size and surface properties of the carrier particles to be labelled and the influence of external factors such as humidity.

The amount of the label particulate material that needs to be attached to the surface of the carrier particles in order to provide a labelled system can be very small. It is possible to achieve high specific activity labelling with 0.03% to 0.05% w/w of Technegas particles based on the weight of the material to be labelled. Thus, it is to be expected that the surface properties of drug particles (for example, hydrophobicity, cohesiveness, etc.), should be unchanged through the labelling procedure and, as a consequence, the properties of the labelled drug within the lung can be assumed to represent the properties of the unlabelled drug.

The present invention in now illustrated but not limited by the following examples.

### EXAMPLE 1

### Wet Labelling Procedure

Technegas particles were produced from a Qados generator as follows:

Sodium pertechnetate in saline solution, collected from a standard elution generator, was loaded into a carbon crucible inside the generator. The generator was sealed and the water in the sodium pertechnetate solution removed by heating the crucible to 70° C. in an argon atmosphere. The chamber was then purged with pure argon to remove the air and water vapour and the crucible resistively heated to 2550° C. to produce Technegas particles suspended in argon.

The Technegas particle stream was slowly bubbled through ether to capture the nanoparticles. The suspension of Technegas particles in ether was admixed with lactose powder (average size 100 μm). Following removal of the ether by evaporation at room temperature, the suspended nanoparticles were deposited on the dried lactose particles demonstrating significant radiolabelling with Technegas particles.

### EXAMPLE 2

### Dry Labelling Procedure with Salbutamol Sulphate

A Technegas particle stream (approximately 100 MBq of activity per litre of argon) was produced as described in Example 1. The drug particles (salbutamol sulphate) with a mass median aerodynamic diameter of about 4 μm were labelled with Technegas particles using the filter assembly apparatus depicted in FIG. 1.

The filter assembly apparatus was connected to the Technegas generator (1). A Technegas particle cloud was drawn out of the generator (1) by means of a vacuum pump (2) and through a chamber (3) containing 100 mg of micronised salbutamol sulphate (4) supported on a piece of filter paper (5). The apparatus was arranged such that the Technegas particle cloud had to pass through the powder bed of salbutamol sulphate (4) and out of the chamber through a filter (6). The salbutamol sulphate was then removed from the die (7), mixed using a metal spatula and replaced in the apparatus Another Technegas particle cloud was then drawn through the salbutamol sulphate. The process was repeated such that the drug was exposed to three Technegas particle clouds.

The radiolabelled salbutamol was filled into gelatine capsules (size 0), loaded into a dry powder inhaler (Aerohaler) and actuated into a multi-stage liquid impinger (MSLI) operated at 60 L/min. This four-stage device separated the emitted dose into different particles size fractions as follows:

Stage 1: 10.0 μm particles

Stage 2: 5.5 μm particles

Stage 3: 3.3 μm particles

Stage 4: 0.8 μm particles

The Aerohaler, induction port (throat) and 4 stages were quantitatively washed with methanol and the resulting solutions analysed by (i) UV spectroscopy to determine the drug concentration and (ii) by scintigraphic analysis to determine the concentration of Technegas particles (the ‘radiolabel’).

To determine the drug concentration, the mass of drug in each methanol solution was calculated (from the sample volume) and the total mass of drug recovered from the Aerohaler, throat and each impinger stage was determined. The mass of drug deposited on the Aerohaler, throat and each impinger stage was then expressed as a % of the total mass of drug recovered (i.e. % of the metered dose).

A gamma camera was used to determine the concentration of Technegas particles in each methanol solution. The gamma camera was used to image each solution, and the images were analysed using a computer program to determine how many radioactive counts were in each solution. The total number of radioactive counts recovered from the Aerohaler, throat and each impinger stage was determined. The number of counts deposited on the Aerohaler, throat and each impinger stage was then expressed as a % of the total number of radioactive counts recovered (i.e. % of the metered dose).

A close agreement between the particle size distributions (PSDs) of the labelled drug and the radioactive nanoparticles was found (see Table 1). This clearly demonstrated efficient radiolabelling of the drug particles with Technegas particles and produced a superior drug/radiolabel match compared to the standard technique.

TABLE 1
PSDs of labelled salbutamol and radiolabel

| | Labelled | |
|---|---|---|
| MSLI | salbutamol | Radiolabel |
| Device | 41.6 | 44.6 |
| Throat | 24.1 | 27.3 |
| Stage 1 | 17.5 | 13.6 |
| Stage 2 | 11.3 | 7.5 |
| Stage 3 | 4.2 | 4.1 |
| Stage 4 | 1.4 | 3.0 |

Data are expressed as % of the metered dose

### EXAMPLE 3

### Dry Labelling Procedure with Budesonide

The dry labelling experiment described above in Example 2 was repeated with another inhalation powder, budesonide, having a mass median aerodynamic diameter of about 3 μm. A close match between the particle size distributions (PSDs) of the labelled drug and the Technegas radiolabel was found (see Table 2). Again, these data clearly demonstrated efficient labelling of the drug particles with Technegas particles.

TABLE 2
PSDs of labelled budesonide and radiolabel

| | Labelled | |
|---|---|---|
| MSLI | budesonide | Radiolabel |
| Device | 40.4 | 43.4 |
| Throat | 22.4 | 19.4 |
| Stage 1 | 28.9 | 23.6 |
| Stage 2 | 1.1 | 2.9 |
| Stage 3 | 1.9 | 4.2 |
| Stage 4 | 5.2 | 6.5 |

Data are expressed as % of the metered dose

### EXAMPLE 4

### Dry Labelling Procedure with a Novel Inhalation Compound

The dry labelling procedure of Example 2 was repeated with a proprietary inhalation compound that could not be adequately radiolabelled using the standard methodology (i.e. the PSD of the radiolabel did not match that of the drug). The results of the Technegas radiolabelling experiment are shown in Table 3.

TABLE 3
PSDs of novel labelled drug and radiolabel

| MSLI | Labelled drug | Radiolabel |
|---|---|---|
| Device | 9.2 | 8.9 |
| Throat | 30.1 | 22.9 |
| Stage 1 | 7.2 | 7.7 |
| Stage 2 | 9.9 | 8.3 |
| Stage 3 | 16.6 | 16.8 |
| Stage 4 | 27.0 | 35.6 |

Data are expressed as % of the metered dose

There was a good match between the particle size distributions (PSDs) of the labelled drug and the radiolabel, indicating efficient labelling of the drug particles with Technegas particles. It should be noted that the Technegas radiolabelling procedure permitted satisfactory labelling of a powder that could not be labelled using conventional labelling techniques.

### EXAMPLE 5

### Controlling the Electrostatic Charge on the Drug Particles to be Dry Labelled

The electrostatic charge (EC) on proprietary drug particles was measured using a Faraday pail linked to a static detector. This device measures the electrostatic charge on samples in coulombs per gram of sample (×10⁻⁹). The particles were then dry labelled and tested as described in Example 2, with the exception that the drug was only exposed to a single Technegas aerosol cloud. Prior to labelling, the drug particles were visibly highly charged and this was confirmed by the magnitude and variability of the EC measurements (mean EC was −0.57 (n=8, range −8.88 to 8.00) nC/g.

The experiments were repeated, this time the drug particles were processed prior to labelling. The drug was sieved through a 150 micron sieve and left in a controlled environment (relative humidity <20%, temperature 21±1° C.) for 24 hours. The EC on the processed drug exhibited a relatively uniform electronegative charge (mean EC was −2.72 (−1.89 to −3.90) nC/g). The PSDs of drug and radiolabel pre- and post-processing are shown on Table 4.

TABLE 4
PSDs of labelled drug and radiolabel, before and after
processing of the drug

| | |
|---|---|
| Pre-processing | Post-processing |

| | | | | |
|---|---|---|---|---|
| | Labelled | | Labelled | |
| MSLI | drug | Radiolabel | drug | Radiolabel |

| | | | | |
|---|---|---|---|---|
| Device | 15.5 | 31.8 | 12.8 | 12.8 |
| Throat | 2.7 | 7.6 | 1.8 | 2.2 |
| Stage 1 | 64.7 | 28.1 | 57 | 56.6 |
| Stage 2 | 7.3 | 10.1 | 12.9 | 12.4 |
| Stage 3 | 5.4 | 12.8 | 8.5 | 8.1 |
| Stage 4 | 3.2 | 8.4 | 5.6 | 6.7 |
| Stage 5 | 1.2 | 1.2 | 1.4 | 1.2 |

Data expressed as % of metered dose

The data clearly show that a good drug/radiolabel match can be obtained if the EC on the drug is controlled prior to labelling. Drug particles with a relatively similar EC of the same polarity will tend to label far more efficiently than particles which exhibit variable EC with fluctuating polarity.

### EXAMPLE 6

### Slow Clearance of Technegas from the Lung

An in vivo study was conducted in 6 healthy volunteers to measure the rate at which Technegas was cleared from the lung. Budesonide was radiolabelled with Technegas as described in Example 3, mixed with lactose (5% w/w) and the resulting powder blend filled into capsules. Subjects inhaled the contents of 2 capsules (total dose of 400 meg drug labelled with 40 MBq of technetium 99 m) via the Aerohaler. Images of the lungs were acquired immediately after dosing and at 0.5, 1.0, 3.0 and 6 hours post dose. Quantitative analysis of the images showed that the Technegas radiolabel was slowly cleared from the lungs, predominantly by mucocillary clearance. The mean clearance profile is shown in FIG. 2. The radiolabel retention data are expressed as % of the initial amount of activity deposited in the lung.

The present invention can provide a simpler and more efficient process for labelling of dry powder formulations for inhalation than has been known hitherto. Additional benefits can include reduced radiation exposure to the operators, since the process is quicker, and better reproducibility.

Another advantage that the labelling system of the present invention can provide when radiolabelled carbon nanoparticles, such as Technegas particles are used is that the radiolabelled particles tend to be less rapidly absorbed through the airway walls into the systemic circulation following deposition in the lung. The radiopharmaceutical commonly used in pulmonary deposition studies, sodium pertechnetate, is rapidly absorbed from the surface of the lung (50% is removed in ˜10 mins). Consequently, scintigraphic images of the lung have to be quickly acquired otherwise the accuracy of the quantification process is adversely affected. The slower clearance rate that is available with radiolabelled carbon nanoparticles means that less of the radiolabel is absorbed whilst the images are being acquired, resulting in improved quantification. Furthermore, the slower clearance rate that tends to be exhibited by carbon nanoparticles means that the technology may be suitable for use with three dimensional imaging techniques such as single photon emission computed tomography (SPECT) where long image acquisition times are required (15 mins of 3 mins for planar imaging).

1. A dry powder composition which comprises smaller label particles which are attached to larger particles.

2. A composition according to claim 1 which is adapted for delivery to the lung.

3. A composition according to claim 1 which is adapted for delivery to the nasal cavity.

4. A composition according to any one of claims 1 to 3, wherein the label particles are radiolabelled with a gamma emitting radionuclide.

5. A composition according to claim 4, wherein the gamma emitting radionuclide comprises technetium-99m.

6. A composition according to claim 4, wherein the gamma emitting radionuclide comprise iodine-123.

7. A composition according to claim 4, wherein the gamma emitting radionuclide comprises indium-111.

8. A composition according to any one of the preceding claims, wherein the label particles have a mean size between 1 and 200 nm.

9. A composition according to claim 8, wherein the mean size of the label particles is less than 100 nm.

10. A composition according to any one of claims 1 to 3, wherein the label particles comprise a core of a gamma emitting radionuclide and a shell of a non-radioactive material.

11. A composition according to claim 10, wherein the shell is made of carbon.

12. A composition according to claim 10 or 11, wherein the shell completely encases the core of the gamma emitting radionuclide.

13. A composition according to any one of claims 10 to 12, wherein the gamma emitting radionuclide comprises iodine-123.

14. A composition according to any one of claims 10 to 12, wherein the gamma emitting radionuclide comprises indium-111.

15. A composition according to any one of claims 10 to 12, wherein the gamma emitting radionuclide comprises technetium-99m.

16. A composition according to claim 11, wherein the label particles are the Technegas particles produced by a Technegas generator (Qados).

17. A composition according to claim 11, wherein the label particles are the Pertechnegas particles produced by a Technegas generator (Qados).

18. A composition according to claim 11, wherein the label particles are a mixture of Technegas and Pertechnegas particles produced by a Technegas generator (Qados).

19. A composition according to any one of the preceding claims, wherein the larger particles are drug particles.

20. A composition according to claim 19, wherein the drug particles have a mean size between 500 nm and 100 μm.

21. A composition according to claim 19 or 20, wherein the drug is a bronchodilator.

22. A composition according to claim 21, wherein the bronchodilator is a beta-2-agonist or an anticholingeric.

23. A composition according to claim 22, wherein the beta 2-agonist is salbutamol and the anticholingeric is ipratropium bromide.

24. A composition according to claim 19 or 20, wherein the drug is an anti-inflammatory agent.

25. A composition according to claim 24, wherein the drug is a corticosteroid or a non-steroidal compound.

26. A composition according to claim 25, wherein the corticosteroid is budesonide and the non-steroidal compound is sodium cromoglycate or nedocromil sodium.

27. A composition according to claim 19 or 20, wherein the drug is selected from polypeptides, insulin, growth hormones, parathyroid hormone, calcitonin, octreotide, lenprolide, leutenising hormone releasing hormone, and interferons-alpha, beta and gamma.

28. A composition according to claim 19 or 20, wherein the drug is selected from the group of aerosolised antibiotics, anti-infectives, anti-virals, mucolytic agents, alpha-1-antiypsin, diuretics, phospodiesterase inhibitors and leukotriene antagonists.

29. A composition according to claim 28, wherein the aerosolised antibiotic is gentamicin, the anti-infective is pentamidine, the anti-viral is rimantadine, the mucolytic agent is rhDNase and the diuretic is frusemide.

30. A method for radiolabelling a material composed of larger particles wherein smaller labelled particles are adhered to the larger particles to form a labelled composition.

31. A method according to claim 30, wherein the labelled composition that is formed is a dry powder composition as claimed in any one of claims 1 to 29.

32. A method for radiolabelling a dry powder composition for inhalation, wherein smaller labelled nanoparticles are mixed with larger particles of drug or an excipient.

33. A method for evaluating the distribution of a material inhaled into the respiratory tract, said method comprising using a dry powder composition comprising smaller label particles that are attached to larger particles of the material to be evaluated.

34. A method according to claim 33, wherein the dry powder composition is delivered to the respiratory tract with a dry powder delivery device.

35. A method according to claim 33 or 34, wherein the dry powder composition is a dry powder composition according to any one of claims 1 to 29.

36. The use of a dry powder composition comprising smaller label particles that are attached to larger particles to evaluate the distribution of the larger particles in the respiratory tract following inhalation of the dry powder composition.

37. The use according to claim 36, wherein the dry powder composition is as claimed in any one of claims 1 to 29.

38. The use of smaller label particles and larger particles of a material to be evaluated in the manufacture of a dry powder composition to evaluate the distribution of the larger particles in the respiratory tract following inhalation of the dry powder composition.

39. The use according to claim 38, wherein the dry powder composition is as claimed in any one of claims 1 to 29.

40. A dry powder composition comprising label particles that comprise a core of a gamma emitting radionuclide and a shell of a non-radioactive material that are attached to a second particulate material.

41. A composition according to claim 40, wherein the shell of the label particles is made of carbon.

42. A composition according to claim 40 or 41, wherein the shell of the label particles completely encases the core of the gamma emitting radionuclide.

43. A composition according to any one of claims 40 to 42, wherein the gamma emitting radionuclide comprises iodine-123.

44. A composition according to any one of claims 40 to 42, wherein the gamma emitting radionuclide comprises indium-111.

45. A composition according to any one of claims 40 to 42, wherein the gamma emitting radionuclide comprises technetium-99m.

46. A composition according to claim 41, wherein the label particles are the Technegas particles produced by a Technegas generator (Qados).

47. A composition according to claim 41, wherein the label particles are the Pertechnegas particles produced by a Technegas generator (Qados).

48. A composition according to claim 41, wherein the label particles are a mixture of Technegas and Pertechnegas particles produced by a Technegas generator (Qados).

49. A composition according to any one of claims 40 to 48, wherein the second particulate material is a drug.

50. A method for radiolabelling a particulate material wherein label particles comprising a core of a gamma emitting radionuclide and a shell of a non-radioactive material are adhered to the particles of the particulate material to form a labelled composition.

51. A method according to claim 50, wherein the labelled composition that is formed is a dry powder composition as claimed in any one of claims 40 to 49.

52. A method for radiolabelling a dry powder composition for inhalation, wherein label particles comprising a core of a gamma emitting radionuclide and a shell of a non-radioactive material are mixed with particles of drug or an excipient.

53. A method for evaluating the distribution of a material inhaled into the respiratory tract, said method comprising using a dry powder composition according to any one of claims 40 to 49.

54. A method according to claim 53, wherein the dry powder composition is delivered to the respiratory tract with a dry powder delivery device.

55. A method for evaluating the distribution of a particulate material inhaled into the respiratory tract, said method comprising associating with said particulate material label particles as defined in any one of claims 40 to 49.

56. The use of label particles comprising a core of a gamma emitting radionuclide and a shell of a non-radioactive material to evaluate the distribution of a second particulate material in the respiratory tract.

57. The use of label particles comprising a core of a gamma emitting radionuclide and a shell of a non-radioactive material in the manufacture of a dry powder composition comprising said label particles attached to a second particulate material to evaluate the distribution of the second particulate material in the respiratory tract following inhalation of the dry powder composition.

58. The use of claim 56 or 57, wherein the shell of the label particles is made of carbon.

59. The use of any one of claims 56 to 58, wherein the shell of the label particles completely encases the core of the gamma emitting radionuclide.

60. The use of any one of claims 56 to 59, wherein the gamma emitting radionuclide comprises iodine-123.

61. The use of any one of claims 56 to 59, wherein the gamma emitting radionuclide comprises indium-111.

62. The use of any one of claims 56 to 59, wherein the gamma emitting radionuclide comprises technetium-99m.

63. The use of claim 58, wherein the label particles are the Technegas particles produced by a Technegas generator (Qados).

64. The use of claim 58, wherein the label particles are the Pertechnegas particles produced by a Technegas generator (Qados).

65. The use of claim 58, wherein the label particles are a mixture of Technegas and Pertechnegas particles produced by a Technegas generator (Qados).

66. The use of claim 58, wherein the second particulate material is a drug.
